(19) United States
(12) Patent Application Publication
(10) Pub. No.: US 2024/0207303 A1
(43) Pub. Date: Jun. 27, 2024

(54) mNA-based Delivery of Botulinum Toxin

(71) Applicants: Sarfaraz K. Niazi, Deerfield, IL (US); Matthias Magoola, Kampala (UG)

(72) Inventors: Sarfaraz K. Niazi, Deerfield, IL (US); Matthias Magoola, Kampala (UG)

(73) Assignee: RNA Therapeutics, Inc., Deerfield, IL (US)

(21) Appl. No.: 18/503,559

(22) Filed: Nov. 7, 2023

(51) Int. Cl. A61K 31/7105; A61K 9/51; A61K 38/48; C12N 15/88

(52) U.S. Cl. CPC A61K 31/7105; A61K 9/5192; A61K 38/4893; C12N 15/88

(57) ABSTRACT

The present invention relates to a composition for expressing botulinum toxin in target tissue by injecting or topically applying an mRNA product capable of expressing the toxin.

### SUMMARY OF THE INVENTION

The invention relates to the technical field of biotechnology, to a therapeutic delivery of botulinum toxin using mRNA instead of naturally extracted toxin from Clostridium botulinum. Additionally, the lipid nanoparticle holding the mRNA can be introduced in the specific area of the body where an action is desired by topical application, in addition to the traditional injection method.

### BACKGROUND OF THE INVENTION

Botulinum toxin has a wide range of applications in medical and cosmetic fields due to its ability to temporarily paralyze muscles and block nerve signals. In cosmetic procedures, it is extensively utilized for wrinkle reduction in areas like the forehead, around the eyes, and between the eyebrows. Botulinum toxin also finds applications in smoothing neck bands, enhancing lips, and lifting eyebrows. On the medical front, it treats conditions like muscle spasms, chronic migraines, hyperhidrosis (excessive sweating), overactive bladder, and eye muscle disorders, among others. Botulinum toxin is also used for pain management, spasticity management in conditions like cerebral palsy, and even in ophthalmology for dry eye syndrome management. In cosmetic dentistry, botulinum toxin can lower the upper lip to reduce the visibility of a gummy smile. It's essential to administer botulinum toxin under the care of trained medical professionals to ensure safety and efficacy, as its effects are temporary and require periodic treatments.

The toxic nature of the toxin requires extreme care in its manufacturing and administration, a challenge that can be removed by expressing the toxin in the body through mRNA instead of injecting it. Also, when the mRNA is formulated in lipid nanoparticles, it is possible to administer the product topically despite the possibility of low absorption since the dose requirement for the toxin is in nanograms.

Botulinum toxin is produced by the bacterium Clostridium botulinum. It elaborates on eight antigenically distinguishable exotoxins (UniProt. A: P0DPI0; B: P10844; C1: P18640; C2: Q9Z2I6; D: P19321; E: Q00496; G: Q60393). Type A is the most potent toxin, followed by types B and F. Types A, B, and E are commonly associated with systemic botulism in humans. All botulinum neurotoxins are produced as relatively inactive, single polypeptide chains with a molecular mass of about 150 kDa and a high degree of amino acid sequence homology among the toxin types. The polypeptide chain consists of a heavy (H) chain and a light (L) chain of roughly 100 and 50 kDa, respectively, linked by a disulfide bond. The botulinum neurotoxin complex is also associated with other nontoxic proteins, which may have hemagglutinating properties.

All serotypes interfere with neural transmission by blocking the release of acetylcholine, the principal neurotransmitter at the neuromuscular junction, causing muscle paralysis. The weakness induced by injection with botulinum toxin A usually lasts about three months. Botulinum toxins now play a very significant role in the management of a wide variety of medical conditions, especially strabismus and focal dystonias, hemifacial spasm, various spastic movement disorders, headaches, hypersalivation, hyperhidrosis, and some chronic conditions that respond only partially to medical treatment. The list of possible new indications is rapidly expanding. Cosmetic applications include correcting lines, creases, and wrinkles all over the face, chin, neck, and chest to dermatological applications such as hyperhidrosis. Injections with botulinum toxin are generally well tolerated, and side effects are few. A precise knowledge and understanding of the functional anatomy of the mimetic muscles is necessary to use botulinum toxins in clinical practice correctly.

Botulinum toxins act at four different sites in the body: The neuromuscular junction, autonomic ganglia, postganglionic parasympathetic nerve endings, and postganglionic sympathetic nerve endings that release acetylcholine. The toxin's heavy (H) chain binds selectively and irreversibly to high-affinity receptors at the presynaptic surface of cholinergic neurons, and the toxin-receptor complex is taken up into the cell by endocytosis. The disulfide bond between the two chains is cleaved, and the toxin escapes into the cytoplasm. The light (L) chain interacts with different proteins (synaptosome-associated protein (SNAP) 25, vesicle-associated membrane protein, and syntaxin) in the nerve terminals to prevent fusion of acetylcholine vesicles with the cell membrane. The peak of the paralytic effect occurs four to seven days after injection. Doses of all commercially available botulinum toxins are expressed in terms of units of biologic activity. One unit of botulinum toxin corresponds to the calculated median intraperitoneal lethal dose (LD50) in female Swiss-Webster mice. The dose of the neurotoxin administered ranges from 5-25 nanograms. The affected nerve terminals do not degenerate, but the blockage of neurotransmitter release is irreversible. The function can be recovered by sprouting nerve terminals and forming new synaptic contacts; this usually takes two to three months.

Botulinum toxin induces weakness of striated muscles by inhibiting transmission of alpha motor neurons at the neuromuscular junction. This has led to its use in conditions with muscular overactivity, such as dystonia. Transmission is also inhibited at gamma neurons in muscle spindles, which may alter reflex overactivity. The toxin also inhibits the release of acetylcholine in all parasympathetic and cholinergic postganglionic sympathetic neurons. This has generated interest in its use as a treatment for overactive smooth muscles (for example, in achalasia) or abnormal activity of glands (for example, hyperhidrosis).

The toxin requires 24-72 hours to take effect, reflecting the time necessary to disrupt the synaptosome process. In rare circumstances, some individuals may require as many as five days to observe the full effect. Peaking at about ten days, the effect of botulinum toxin lasts nearly 8-12 weeks.

Type A botulinum toxin has widened its clinical range of applications, but the risk of developing antibodies limits the repeated use of high-dose injection. Other serotypes of botulinum toxin are being investigated as useful alternatives. Botulinum toxin type F differs from type A, mainly by its lower potency, efficacy, and shorter duration of action, and it blocks a different SNARE protein compared to type A toxin. Therefore, a combination of toxins A and F has been suggested to reduce the total units and overall antigenic dose.

mRNA can express proteins depending on its sequence and adaptability to interact with ribosomes. The inherent adaptability of nucleotide products has sparked interest in their application to express therapeutic proteins instead of injecting recombinantly produced proteins or natural proteins.

### DETAILS OF THE INVENTION

To make the objects, technical solutions, and advantages of the embodiments of the present invention more apparent, the technical solutions in the embodiments of the present invention will be clearly and completely described below, but not all embodiments of the present invention. Thus, the following detailed description of the embodiments of the present invention is not intended to limit the scope of the invention, as claimed, but is merely representative of selected embodiments of the invention. All other embodiments, which can be derived by a person skilled in the art from the embodiments given herein without making any creative effort, shall fall within the protection scope of the present invention.

mRNA molecules are designed to encode for specific antigens of HIV and HPV. The mRNA is structurally composed of a 5′UTR, a signal peptide for efficient translation, the open reading frame that encodes the antigen, a 3′UTR for stability, and a polyA tail. This structure ensures efficient translation and antigen presentation for an immune response. Using pseudouridine in the mRNA is a modification to avoid innate immune sensing and enhance translation efficiency. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs. However, unlike the ORF, those elements need not necessarily be present in a product of the present disclosure.

The nucleotide modifications within the mRNA are crucial because they help to evade the host's innate immune responses, which can often degrade mRNA before it achieves its purpose. These modifications can enhance the translational capacity and stability of the mRNA, leading to higher and more prolonged protein expression of the product antigen within the body. As a result, these products can induce robust and sustained immune responses, which are critical for preventive and therapeutic product strategies.

The LNP delivery system is an essential component of the invention, allowing for the encapsulation and delivery of the mRNA into human cells. The LNPs are designed to include an ionizable cationic lipid for effective delivery, a non-cationic lipid for structural stability, and a PEGylated lipid to extend circulation time in the bloodstream. Lyophilization of the LNP is included to enhance stability and shelf-life, making the product suitable for distribution and storage.

In some respects, the present disclosure provides compositions of introducing botulinum toxin in a target tissue.

Lyophilization of LNP formulation extends the storage life of the product. It allows storage at higher temperatures, making it an essential consideration for product distribution.

In the first embodiment, the present invention uses an mRNA molecule to express botulinum toxin in the body.

In the second embodiment, the present invention provides mRNA molecules formulated in a lipid nanoparticle that can be administered by injection or topical application.

In a third embodiment, the present invention provides a mRNA molecule comprising a 5′UTR element, a signal peptide element, an open reading frame corresponding to the expressed proteins, a 3′ UTR element, and a polyA tail element linked together in that order.

In a fourth embodiment, the uracil, cytosine, or adenine nucleotides of the therapeutic mRNA molecule contain a modifying group that includes at least one of pseudouridine, N1-methyl pseudouridine, N1-ethylpseudouridine, 5-methylcytosine, 2-thiouridine, 5-methoxyuridine, or N1-methyladenosine. RNA sequences are modified by replacing uridine (U) with pseudouridine (Ψ).

In a fifth embodiment, the present invention claims an LNP formulation that comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

In a sixth embodiment, this invention relates to the field of biotechnology, where the botulinum toxic is expressed in the body by introducing an mRNA sequence of nucleotides into the body comprising SEQ No. 1 (U replaced with T:

GAGAATAAACTAGTATTCTTCTGGTCCCCACAGACTCAGAGAGAACCCG
CCACCATGCCCTTCGTGAACAAGCAGTTCAACTACAAGGACCCCGTGAA
CGGCGTGGACATCGCCTACATCAAGATCCCCAACGTGGGCCAGATGCAG
CCCGTGAAGGCCTTCAAGATCCACAACAAGATCTGGGTGATCCCCGAGA
GGGACACCTTCACCAACCCCGAGGAGGGCGACCTGAACCCCCCCCCCGA
GGCCAAGCAGGTGCCCGTGAGCTACTACGACAGCACCTACCTGAGCACC
GACAACGAGAAGGACAACTACCTGAAGGGCGTGACCAAGCTGTTCGAGA
GGATCTACAGCACCGACCTGGGCAGGATGCTGCTGACCAGCATCGTGAG
GGGCATCCCCTTCTGGGGCGGCAGCACCATCGACACCGAGCTGAAGGTG
ATCGACACCAACTGCATCAACGTGATCCAGCCCGACGGCAGCTACAGGA
GCGAGGAGCTGAACCTGGTGATCATCGGCCCCAGCGCCGACATCATCCA
GTTCGAGTGCAAGAGCTTCGGCCACGAGGTGCTGAACCTGACCAGGAAC
GGCTACGGCAGCACCCAGTACATCAGGTTCAGCCCCGACTTCACCTTCG
GCTTCGAGGAGAGCCTGGAGGTGGACACCAACCCCCTGCTGGGCGCCGG
CAAGTTCGCCACCGACCCCGCCGTGACCCTGGCCCACGAGCTGATCCAC
GCCGGCCACAGGCTGTACGGCATCGCCATCAACCCCAACAGGGTGTTCA
AGGTGAACACCAACGCCTACTACGAGATGAGCGGCCTGGAGGTGAGCTT
CGAGGAGCTGAGGACCTTCGGCGGCCACGACGCCAAGTTCATCGACAGC
CTGCAGGAGAACGAGTTCAGGCTGTACTACTACAACAAGTTCAAGGACA
TCGCCAGCACCCTGAACAAGGCCAAGAGCATCGTGGGCACCACCGCCAG
CCTGCAGTACATGAAGAACGTGTTCAAGGAGAAGTACCTGCTGAGCGAG
GACACCAGCGGCAAGTTCAGCGTGGACAAGCTGAAGTTCGACAAGCTGT
ACAAGATGCTGACCGAGATCTACACCGAGGACAACTTCGTGAAGTTCTT
CAAGGTGCTGAACAGGAAGACCTACCTGAACTTCGACAAGGCCGTGTTC
AAGATCAACATCGTGCCCAAGGTGAACTACACCATCTACGACGGCTTCA
ACCTGAGGAACACCAACCTGGCCGCCAACTTCAACGGCCAGAACACCGA
GATCAACAACATGAACTTCACCAAGCTGAAGAACTTCACCGGCCTGTTC
GAGTTCTACAAGCTGCTGTGCGTGAGGGGCATCATCACCAGCAAGACCA
AGAGCCTGGACAAGGGCTACAACAAGGCCCTGAACGACCTGTGCATCAA
GGTGAACAACTGGGACCTGTTCTTCAGCCCCAGCGAGGACAACTTCACC
AACGACCTGAACAAGGGCGAGGAGATCACCAGCGACACCAACATCGAGG
CCGCCGAGGAGAACATCAGCCTGGACCTGATCCAGCAGTACTACCTGAC
CTTCAACTTCGACAACGAGCCCGAGAACATCAGCATCGAGAACCTGAGC
AGCGACATCATCGGCCAGCTGGAGCTGATGCCCAACATCGAGAGGTTCC
CCAACGGCAAGAAGTACGAGCTGGACAAGTACACCATGTTCCACTACCT
GAGGGCCCAGGAGTTCGAGCACGGCAAGAGCAGGATCGCCCTGACCAAC
AGCGTGAACGAGGCCCTGCTGAACCCCAGCAGGGTGTACACCTTCTTCA
GCAGCGACTACGTGAAGAAGGTGAACAAGGCCACCGAGGCCGCCATGTT
CCTGGGCTGGGTGGAGCAGCTGGTGTACGACTTCACCGACGAGACCAGC
GAGGTGAGCACCACCGACAAGATCGCCGACATCACCATCATCATCCCCT
ACATCGGCCCCGCCCTGAACATCGGCAACATGCTGTACAAGGACGACTT
CGTGGGCGCCCTGATCTTCAGCGGCGCCGTGATCCTGCTGGAGTTCATC
CCCGAGATCGCCATCCCCGTGCTGGGCACCTTCGCCCTGGTGAGCTACA
TCGCCAACAAGGTGCTGACCGTGCAGACCATCGACAACGCCCTGAGCAA
GAGGAACGAGAAGTGGGACGAGGTGTACAAGTACATCGTGACCAACTGG
CTGGCCAAGGTGAACACCCAGATCGACCTGATCAGGAAGAAGATGAAGG
AGGCCCTGGAGAACCAGGCCGAGGCCACCAAGGCCATCATCAACTACCA
GTACAACCAGTACACCGAGGAGGAGAAGAACAACATCAACTTCAACATC
GACGACCTGAGCAGCAAGCTGAACGAGAGCATCAACAAGGCCATGATCA
ACATCAACAAGTTCCTGAACCAGTGCAGCGTGAGCTACCTGATGAACAG
CATGATCCCCTACGGCGTGAAGAGGCTGGAGGACTTCGACGCCAGCCTG
AAGGACGCCCTGCTGAAGTACATCTACGACAACAGGGGCACCCTGATCG
GCCAGGTGGACAGGCTGAAGGACAAGGTGAACAACACCCTGAGCACCGA
CATCCCCTTCCAGCTGAGCAAGTACGTGGACAACCAGAGGCTGCTGAGC
ACCTTCACCGAGTACATCAAGAACATCATCAACACCAGCATCCTGAACC
TGAGGTACGAGAGCAACCACCTGATCGACCTGAGCAGGTACGCCAGCAA
GATCAACATCGGCAGCAAGGTGAACTTCGACCCCATCGACAAGAACCAG
ATCCAGCTGTTCAACCTGGAGAGCAGCAAGATCGAGGTGATCCTGAAGA
ACGCCATCGTGTACAACAGCATGTACGAGAACTTCAGCACCAGCTTCTG
GATCAGGATCCCCAAGTACTTCAACAGCATCAGCCTGAACAACGAGTAC
ACCATCATCAACTGCATGGAGAACAACAGCGGCTGGAAGGTGAGCCTGA
ACTACGGCGAGATCATCTGGACCCTGCAGGACACCCAGGAGATCAAGCA
GAGGGTGGTGTTCAAGTACAGCCAGATGATCAACATCAGCGACTACATC
AACAGGTGGATCTTCGTGACCATCACCAACAACAGGCTGAACAACAGCA
AGATCTACATCAACGGCAGGCTGATCGACCAGAAGCCCATCAGCAACCT
GGGCAACATCCACGCCAGCAACAACATCATGTTCAAGCTGGACGGCTGC
AGGGACACCCACAGGTACATCTGGATCAAGTACTTCAACCTGTTCGACA
AGGAGCTGAACGAGAAGGAGATCAAGGACCTGTACGACAACCAGAGCAA
CAGCGGCATCCTGAAGGACTTCTGGGGCGACTACCTGCAGTACGACAAG
CCCTACTACATGCTGAACCTGTACGACCCCAACAAGTACGTGGACGTGA
ACAACGTGGGCATCAGGGGCTACATGTACCTGAAGGGCCCCAGGGGCAG
CGTGATGACCACCAACATCTACCTGAACAGCAGCCTGTACAGGGGCACC
AAGTTCATCATCAAGAAGTACGCCAGCGGCAACAAGGACAACATCGTGA
GGAACAACGACAGGGTGTACATCAACGTGGTGGTGAAGAACAAGGAGTA
CAGGCTGGCCACCAACGCCAGCCAGGCCGGCGTGGAGAAGATCCTGAGC
GCCCTGGAGATCCCCGACGTGGGCAACCTGAGCCAGGTGGTGGTGATGA
AGAGCAAGAACGACCAGGGCATCACCAACAAGTGCAAGATGAACCTGCA
GGACAACAACGGCAACGACATCGGCTTCATCGGCTTCCACCAGTTCAAC
AACATCGCCAAGCTGGTGGCCAGCAACTGGTACAACAGGCAGATCGAGA
GGAGCAGCAGGACCCTGGGCTGCAGCTGGGAGTTCATCCCCGTGGACGA
CGGCTGGGGCGAGAGGCCCCTGTGACTCGAGCTGGTACTGCATGCACGC
AATGCTAGCTGCCCCTTTCCCGTCCTGGGTACCCCGAGTCTCCCCCGAC
CTCGGGTCCCAGGTATGCTCCCACCTCCACCTGCCCCACTCACCACCTC
TGCTAGTTCCAGACACCTCCCAAGCACGCAGCAATGCAGCTCAAAACGC
TTAGCCTAGCCACACCCCCACGGGAAACAGCAGTGATTAACCTTTAGCA
ATAAACGAAAGTTTAACTAAGCTATACTAACCCCAGGGTTGGTCAATTT
CGTGCCAGCCACACCCTGGAGCTAGCAAAAAAAAAAAAAAAAAAAAAAA
AAAAAAAGCATATGACTAAAAAAAAAAAAAAAAAAAAAAAAAAAAAAAA
AAAAAAAAAAAAAAAAAAAAAAAAAAAAAAAAAAAAAA.

1. A composition for administering botulinum toxin in a target tissue comprising injecting or topically applying messenger RNA (mRNA) designed to express botulinum toxin.

2. The composition of claim 1, wherein the mRNA molecules are enclosed in lipid nanoparticles further comprising an ionizable cationic lipid, a non-cationic lipid, and a PEG-modified lipid.

3. The composition of claim 1, wherein the lipid nanoparticle formulation comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

4. The composition of claim 2, wherein the lipid nanoparticles are lyophilized.

5. The composition of claim 1, wherein the mRNA molecules comprise a 5′UTR element, a signal peptide element, an open reading frame corresponding to the expressed proteins, a 3′ UTR element, and a polyA tail linked together in that order.

6. The composition of claim 5, wherein the mRNA comprises
GAGAAUAAACUAGUAUUCUUCUGGUCCCCACAGACUCAGAGAGAACCCG
CCACCAUGCCCUUCGUGAACAAGCAGUUCAACUACAAGGACCCCGUGAA
CGGCGUGGACAUCGCCUACAUCAAGAUCCCCAACGUGGGCCAGAUGCAG
CCCGUGAAGGCCUUCAAGAUCCACAACAAGAUCUGGGUGAUCCCCGAGA
GGGACACCUUCACCAACCCCGAGGAGGGCGACCUGAACCCCCCCCCCGA
GGCCAAGCAGGUGCCCGUGAGCUACUACGACAGCACCUACCUGAGCACC
GACAACGAGAAGGACAACUACCUGAAGGGCGUGACCAAGCUGUUCGAGA
GGAUCUACAGCACCGACCUGGGCAGGAUGCUGCUGACCAGCAUCGUGAG
GGGCAUCCCCUUCUGGGGCGGCAGCACCAUCGACACCGAGCUGAAGGUG
AUCGACACCAACUGCAUCAACGUGAUCCAGCCCGACGGCAGCUACAGGA
GCGAGGAGCUGAACCUGGUGAUCAUCGGCCCCAGCGCCGACAUCAUCCA
GUUCGAGUGCAAGAGCUUCGGCCACGAGGUGCUGAACCUGACCAGGAAC
GGCUACGGCAGCACCCAGUACAUCAGGUUCAGCCCCGACUUCACCUUCG
GCUUCGAGGAGAGCCUGGAGGUGGACACCAACCCCCUGCUGGGCGCCGG
CAAGUUCGCCACCGACCCCGCCGUGACCCUGGCCCACGAGCUGAUCCAC
GCCGGCCACAGGCUGUACGGCAUCGCCAUCAACCCCAACAGGGUGUUCA
AGGUGAACACCAACGCCUACUACGAGAUGAGCGGCCUGGAGGUGAGCUU
CGAGGAGCUGAGGACCUUCGGCGGCCACGACGCCAAGUUCAUCGACAGC
CUGCAGGAGAACGAGUUCAGGCUGUACUACUACAACAAGUUCAAGGACA
UCGCCAGCACCCUGAACAAGGCCAAGAGCAUCGUGGGCACCACCGCCAG
CCUGCAGUACAUGAAGAACGUGUUCAAGGAGAAGUACCUGCUGAGCGAG
GACACCAGCGGCAAGUUCAGCGUGGACAAGCUGAAGUUCGACAAGCUGU
ACAAGAUGCUGACCGAGAUCUACACCGAGGACAACUUCGUGAAGUUCUU
CAAGGUGCUGAACAGGAAGACCUACCUGAACUUCGACAAGGCCGUGUUC
AAGAUCAACAUCGUGCCCAAGGUGAACUACACCAUCUACGACGGCUUCA
ACCUGAGGAACACCAACCUGGCCGCCAACUUCAACGGCCAGAACACCGA
GAUCAACAACAUGAACUUCACCAAGCUGAAGAACUUCACCGGCCUGUUC
GAGUUCUACAAGCUGCUGUGCGUGAGGGGCAUCAUCACCAGCAAGACCA
AGAGCCUGGACAAGGGCUACAACAAGGCCCUGAACGACCUGUGCAUCAA
GGUGAACAACUGGGACCUGUUCUUCAGCCCCAGCGAGGACAACUUCACC
AACGACCUGAACAAGGGCGAGGAGAUCACCAGCGACACCAACAUCGAGG
CCGCCGAGGAGAACAUCAGCCUGGACCUGAUCCAGCAGUACUACCUGAC
CUUCAACUUCGACAACGAGCCCGAGAACAUCAGCAUCGAGAACCUGAGC
AGCGACAUCAUCGGCCAGCUGGAGCUGAUGCCCAACAUCGAGAGGUUCC
CCAACGGCAAGAAGUACGAGCUGGACAAGUACACCAUGUUCCACUACCU
GAGGGCCCAGGAGUUCGAGCACGGCAAGAGCAGGAUCGCCCUGACCAAC
AGCGUGAACGAGGCCCUGCUGAACCCCAGCAGGGUGUACACCUUCUUCA
GCAGCGACUACGUGAAGAAGGUGAACAAGGCCACCGAGGCCGCCAUGUU
CCUGGGCUGGGUGGAGCAGCUGGUGUACGACUUCACCGACGAGACCAGC
GAGGUGAGCACCACCGACAAGAUCGCCGACAUCACCAUCAUCAUCCCCU
ACAUCGGCCCCGCCCUGAACAUCGGCAACAUGCUGUACAAGGACGACUU
CGUGGGCGCCCUGAUCUUCAGCGGCGCCGUGAUCCUGCUGGAGUUCAUC
CCCGAGAUCGCCAUCCCCGUGCUGGGCACCUUCGCCCUGGUGAGCUACA
UCGCCAACAAGGUGCUGACCGUGCAGACCAUCGACAACGCCCUGAGCAA
GAGGAACGAGAAGUGGGACGAGGUGUACAAGUACAUCGUGACCAACUGG
CUGGCCAAGGUGAACACCCAGAUCGACCUGAUCAGGAAGAAGAUGAAGG
AGGCCCUGGAGAACCAGGCCGAGGCCACCAAGGCCAUCAUCAACUACCA
GUACAACCAGUACACCGAGGAGGAGAAGAACAACAUCAACUUCAACAUC
GACGACCUGAGCAGCAAGCUGAACGAGAGCAUCAACAAGGCCAUGAUCA
ACAUCAACAAGUUCCUGAACCAGUGCAGCGUGAGCUACCUGAUGAACAG
CAUGAUCCCCUACGGCGUGAAGAGGCUGGAGGACUUCGACGCCAGCCUG
AAGGACGCCCUGCUGAAGUACAUCUACGACAACAGGGGCACCCUGAUCG
GCCAGGUGGACAGGCUGAAGGACAAGGUGAACAACACCCUGAGCACCGA
CAUCCCCUUCCAGCUGAGCAAGUACGUGGACAACCAGAGGCUGCUGAGC
ACCUUCACCGAGUACAUCAAGAACAUCAUCAACACCAGCAUCCUGAACC
UGAGGUACGAGAGCAACCACCUGAUCGACCUGAGCAGGUACGCCAGCAA
GAUCAACAUCGGCAGCAAGGUGAACUUCGACCCCAUCGACAAGAACCAG
AUCCAGCUGUUCAACCUGGAGAGCAGCAAGAUCGAGGUGAUCCUGAAGA
ACGCCAUCGUGUACAACAGCAUGUACGAGAACUUCAGCACCAGCUUCUG
GAUCAGGAUCCCCAAGUACUUCAACAGCAUCAGCCUGAACAACGAGUAC
ACCAUCAUCAACUGCAUGGAGAACAACAGCGGCUGGAAGGUGAGCCUGA
ACUACGGCGAGAUCAUCUGGACCCUGCAGGACACCCAGGAGAUCAAGCA
GAGGGUGGUGUUCAAGUACAGCCAGAUGAUCAACAUCAGCGACUACAUC
AACAGGUGGAUCUUCGUGACCAUCACCAACAACAGGCUGAACAACAGCA
AGAUCUACAUCAACGGCAGGCUGAUCGACCAGAAGCCCAUCAGCAACCU
GGGCAACAUCCACGCCAGCAACAACAUCAUGUUCAAGCUGGACGGCUGC
AGGGACACCCACAGGUACAUCUGGAUCAAGUACUUCAACCUGUUCGACA
AGGAGCUGAACGAGAAGGAGAUCAAGGACCUGUACGACAACCAGAGCAA
CAGCGGCAUCCUGAAGGACUUCUGGGGCGACUACCUGCAGUACGACAAG
CCCUACUACAUGCUGAACCUGUACGACCCCAACAAGUACGUGGACGUGA
ACAACGUGGGCAUCAGGGGCUACAUGUACCUGAAGGGCCCCAGGGGCAG
CGUGAUGACCACCAACAUCUACCUGAACAGCAGCCUGUACAGGGGCACC
AAGUUCAUCAUCAAGAAGUACGCCAGCGGCAACAAGGACAACAUCGUGA
GGAACAACGACAGGGUGUACAUCAACGUGGUGGUGAAGAACAAGGAGUA
CAGGCUGGCCACCAACGCCAGCCAGGCCGGCGUGGAGAAGAUCCUGAGC
GCCCUGGAGAUCCCCGACGUGGGCAACCUGAGCCAGGUGGUGGUGAUGA
AGAGCAAGAACGACCAGGGCAUCACCAACAAGUGCAAGAUGAACCUGCA
GGACAACAACGGCAACGACAUCGGCUUCAUCGGCUUCCACCAGUUCAAC
AACAUCGCCAAGCUGGUGGCCAGCAACUGGUACAACAGGCAGAUCGAGA
GGAGCAGCAGGACCCUGGGCUGCAGCUGGGAGUUCAUCCCCGUGGACGA
CGGCUGGGGCGAGAGGCCCCUGUGACUCGAGCUGGUACUGCAUGCACGC
AAUGCUAGCUGCCCCUUUCCCGUCCUGGGUACCCCGAGUCUCCCCCGAC
CUCGGGUCCCAGGUAUGCUCCCACCUCCACCUGCCCCACUCACCACCUC
UGCUAGUUCCAGACACCUCCCAAGCACGCAGCAAUGCAGCUCAAAACGC
UUAGCCUAGCCACACCCCCACGGGAAACAGCAGUGAUUAACCUUUAGCA
AUAAACGAAAGUUUAACUAAGCUAUACUAACCCCAGGGUUGGUCAAUUU
CGUGCCAGCCACACCCUGGAGCUAGCAAAAAAAAAAAAAAAAAAAAAAA
AAAAAAAGCAUAUGACUAAAAAAAAAAAAAAAAAAAAAAAAAAAAAAAA
AAAAAAAAAAAAAAAAAAAAAAAAAAAAAAAAAAAAAA.

7. The composition of claim 6, wherein the mRNA sequence is modified by replacing uridine (U) with pseudouridine (Ψ), wherever applicable, in the mRNA sequence.


SEQUENCE LISTING (1 sequence)

SEQ ID NO: 1 - RNA, 4350 nt, Synthetic construct
  source 1..4350: mol_type = other RNA
gagaataaactagtattcttctggtccccacagactcagagagaacccgccaccatgcccttcgtgaacaagcagttcaactacaaggaccccgtgaacggcgtggacatcgcctacatcaagatccccaacgtgggccagatgcagcccgtgaaggccttcaagatccacaacaagatctgggtgatccccgagagggacaccttcaccaaccccgaggagggcgacctgaaccccccccccgaggccaagcaggtgcccgtgagctactacgacagcacctacctgagcaccgacaacgagaaggacaactacctgaagggcgtgaccaagctgttcgagaggatctacagcaccgacctgggcaggatgctgctgaccagcatcgtgaggggcatccccttctggggcggcagcaccatcgacaccgagctgaaggtgatcgacaccaactgcatcaacgtgatccagcccgacggcagctacaggagcgaggagctgaacctggtgatcatcggccccagcgccgacatcatccagttcgagtgcaagagcttcggccacgaggtgctgaacctgaccaggaacggctacggcagcacccagtacatcaggttcagccccgacttcaccttcggcttcgaggagagcctggaggtggacaccaaccccctgctgggcgccggcaagttcgccaccgaccccgccgtgaccctggcccacgagctgatccacgccggccacaggctgtacggcatcgccatcaaccccaacagggtgttcaaggtgaacaccaacgcctactacgagatgagcggcctggaggtgagcttcgaggagctgaggaccttcggcggccacgacgccaagttcatcgacagcctgcaggagaacgagttcaggctgtactactacaacaagttcaaggacatcgccagcaccctgaacaaggccaagagcatcgtgggcaccaccgccagcctgcagtacatgaagaacgtgttcaaggagaagtacctgctgagcgaggacaccagcggcaagttcagcgtggacaagctgaagttcgacaagctgtacaagatgctgaccgagatctacaccgaggacaacttcgtgaagttcttcaaggtgctgaacaggaagacctacctgaacttcgacaaggccgtgttcaagatcaacatcgtgcccaaggtgaactacaccatctacgacggcttcaacctgaggaacaccaacctggccgccaacttcaacggccagaacaccgagatcaacaacatgaacttcaccaagctgaagaacttcaccggcctgttcgagttctacaagctgctgtgcgtgaggggcatcatcaccagcaagaccaagagcctggacaagggctacaacaaggccctgaacgacctgtgcatcaaggtgaacaactgggacctgttcttcagccccagcgaggacaacttcaccaacgacctgaacaagggcgaggagatcaccagcgacaccaacatcgaggccgccgaggagaacatcagcctggacctgatccagcagtactacctgaccttcaacttcgacaacgagcccgagaacatcagcatcgagaacctgagcagcgacatcatcggccagctggagctgatgcccaacatcgagaggttccccaacggcaagaagtacgagctggacaagtacaccatgttccactacctgagggcccaggagttcgagcacggcaagagcaggatcgccctgaccaacagcgtgaacgaggccctgctgaaccccagcagggtgtacaccttcttcagcagcgactacgtgaagaaggtgaacaaggccaccgaggccgccatgttcctgggctgggtggagcagctggtgtacgacttcaccgacgagaccagcgaggtgagcaccaccgacaagatcgccgacatcaccatcatcatcccctacatcggccccgccctgaacatcggcaacatgctgtacaaggacgacttcgtgggcgccctgatcttcagcggcgccgtgatcctgctggagttcatccccgagatcgccatccccgtgctgggcaccttcgccctggtgagctacatcgccaacaaggtgctgaccgtgcagaccatcgacaacgccctgagcaagaggaacgagaagtgggacgaggtgtacaagtacatcgtgaccaactggctggccaaggtgaacacccagatcgacctgatcaggaagaagatgaaggaggccctggagaaccaggccgaggccaccaaggccatcatcaactaccagtacaaccagtacaccgaggaggagaagaacaacatcaacttcaacatcgacgacctgagcagcaagctgaacgagagcatcaacaaggccatgatcaacatcaacaagttcctgaaccagtgcagcgtgagctacctgatgaacagcatgatcccctacggcgtgaagaggctggaggacttcgacgccagcctgaaggacgccctgctgaagtacatctacgacaacaggggcaccctgatcggccaggtggacaggctgaaggacaaggtgaacaacaccctgagcaccgacatccccttccagctgagcaagtacgtggacaaccagaggctgctgagcaccttcaccgagtacatcaagaacatcatcaacaccagcatcctgaacctgaggtacgagagcaaccacctgatcgacctgagcaggtacgccagcaagatcaacatcggcagcaaggtgaacttcgaccccatcgacaagaaccagatccagctgttcaacctggagagcagcaagatcgaggtgatcctgaagaacgccatcgtgtacaacagcatgtacgagaacttcagcaccagcttctggatcaggatccccaagtacttcaacagcatcagcctgaacaacgagtacaccatcatcaactgcatggagaacaacagcggctggaaggtgagcctgaactacggcgagatcatctggaccctgcaggacacccaggagatcaagcagagggtggtgttcaagtacagccagatgatcaacatcagcgactacatcaacaggtggatcttcgtgaccatcaccaacaacaggctgaacaacagcaagatctacatcaacggcaggctgatcgaccagaagcccatcagcaacctgggcaacatccacgccagcaacaacatcatgttcaagctggacggctgcagggacacccacaggtacatctggatcaagtacttcaacctgttcgacaaggagctgaacgagaaggagatcaaggacctgtacgacaaccagagcaacagcggcatcctgaaggacttctggggcgactacctgcagtacgacaagccctactacatgctgaacctgtacgaccccaacaagtacgtggacgtgaacaacgtgggcatcaggggctacatgtacctgaagggccccaggggcagcgtgatgaccaccaacatctacctgaacagcagcctgtacaggggcaccaagttcatcatcaagaagtacgccagcggcaacaaggacaacatcgtgaggaacaacgacagggtgtacatcaacgtggtggtgaagaacaaggagtacaggctggccaccaacgccagccaggccggcgtggagaagatcctgagcgccctggagatccccgacgtgggcaacctgagccaggtggtggtgatgaagagcaagaacgaccagggcatcaccaacaagtgcaagatgaacctgcaggacaacaacggcaacgacatcggcttcatcggcttccaccagttcaacaacatcgccaagctggtggccagcaactggtacaacaggcagatcgagaggagcagcaggaccctgggctgcagctgggagttcatccccgtggacgacggctggggcgagaggcccctgtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa